SEQUENCE LISTING (10 sequences)

SEQ ID NO: 1 - protein, 631 aa, Alicyclobacillus acidocaldarius [sequence omitted]

SEQ ID NO: 2 - protein, 631 aa, synthetic construct
  source 1..631: note = SHC#65
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR

SEQ ID NO: 3 - protein, 685 aa, Gluconobacter morbifer
  source 1..685: note = GmoSHC
MSPADISTKSSSFQRLDNMLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 4 - protein, 656 aa, Acetobacter pasteurianus
  source 1..656: note = ApaSHCA
MAADGSALSESRLSSEALDRAVLSAHTALSQAQQDDGHWVYELEADATIPAEYILLEHFMDRIDDALEQKIAIYLRRIQSEEHGGWPLYHNGKFDLSATVKAYFALKAVGDDINAPHMQRAREAILDHGGAERSNVFTRSQLALFGEVPWRATPVMPVELMLLPAKAFFSVWNMSYWSRTVIAPLLVLAALRPVAANPRQVHVRELFVTPPEKVQDWIRGPYRSAWGYVFKGLDSVLRPVVPFIPEKTHKKAIQAALDFIEPRLNGKDGLGAIYPAMANVVMMYRAMGVPDEDPRAKTAWEAVQALIVEKDDEAYCQPCVSPIWDTGLSGHAMIEAASGPNGIAPEKTVAELKKASAWLRSKQILNVKGDWAVRNPNLAPGGWAFQYGNDYYPDVDDTAVVGMLLHREGDPTNAEAIERARTWIVGMQSTDGGWGAFDIDNNKDVLNHIPFADHGALLDPPTADVTARCISFLAQLRNPEDEPVIQRGLEYLRKEQEKDGSWFGRWGTNYIYGTWSALCALNAAGVSHDDPAVVKAVEWLRSVQRADGGWGEGCESYEGGPHGTYGESLPSQTAWAVLGLMAAGRRDDPAVTRGIAWLADQQDANGEWHEDPYNAVGFPKVFYLRYHGYKQFFPLMALARYRNLESSNTRRVSFGF

SEQ ID NO: 5 - protein, 720 aa, Acetobacter pasteurianus
  source 1..720: note = ApaSHC1
MNMASRFSLKKILRSGSDTQGTNVNTLIQSGTSDIVRQKPAPQEPADLSALKAMGNSLTHTLSSACEWLMKQQKPDGHWVGSVGSNASMEAEWCLALWFLGLEDHPLRPRLGKALLEMQRPDGSWGTYYGAGSGDINATVESYAALRSLGYAEDDPAVSKAAAWIISKGGLKNVRVFTRYWLALIGEWPWEKTPNLPPEIIWFPDNFVFSIYNFAQWARATMMPLAILSARRPSRPLRPQDRLDALFPGGRANFDYELPTKEGRDVIADFFRLADKGLHWLQSSFLKRAPSREAAIKYVLEWIIWHQDADGGWGGIQPPWVYGLMALHGEGYQFHHPVMAKALDALNDPGWRHDKGDASWIQATNSPVWDTMLSLMALHDANAEERFTPEMDKALDWLLSRQVRVKGDWSVKLPNTEPGGWAFEYANDRYPDTDDTAVALIAIASCRNRPEWQAKGVEEAIGRGVRWLVAMQSSCGGWGAFDKDNNKSILAKIPFCDFGEALDPPSVDVTAHVLEAFGLLGLPRDLPCIQRGLAYIRKEQDPTGPWFGRWGVNYLYGTGAVLPALAALGEDMTQPYISKACDWLINCQQENGGWGESCASYMEVSSIGHGATTPSQTAWALMGLIAANRPQDYEAIAKGCRYLIDLQEEDGSWNEEEFTGTGFPGYGVGQTIKLDDPAISKRLMQGAELSRAFMLRYDLYRQLFPIIALSRASRLIKLGN

SEQ ID NO: 6 - protein, 684 aa, Bradyrhizobium japonicum
  source 1..684: note = BjaSHC
MTVTSSASARATRDPGNYQTALQSTVRAAADWLIANQKPDGHWVGRAESNACMEAQWCLALWFMGLEDHPLRKRLGQSLLDSQRPDGAWQVYFGAPNGDINATVEAYAALRSLGFRDDEPAVRRAREWIEAKGGLRNIRVFTRYWLALIGEWPWEKTPNIPPEVIWFPLWFPFSIYNFAQWARATLMPIAVLSARRPSRPLPPENRLDALFPHGRKAFDYELPVKAGAGGWDRFFRGADKVLHKLQNLGNRLNLGLFRPAATSRVLEWMIRHQDFDGAWGGIQPPWIYGLMALYAEGYPLNHPVLAKGLDALNDPGWRVDVGDATYIQATNSPVWDTILTLLAFDDAGVLGDYPEAVDKAVDWVLQRQVRVPGDWSMKLPHVKPGGWAFEYANNYYPDTDDTAVALIALAPLRHDPKWKAKGIDEAIQLGVDWLIGMQSQGGGWGAFDKDNNQKILTKIPFCDYGEALDPPSVDVTAHIIEAFGKLGISRNHPSMVQALDYIRREQEPSGPWFGRWGVNYVYGTGAVLPALAAIGEDMTQPYIGRACDWLVAHQQADGGWGESCASYMDVSAVGRGTTTASQTAWALMALLAANRPQDKDAIERGCMWLVERQSAGTWDEPEFTGTGFPGYGVGQTIKLNDPALSQRLMQGPELSRAFMLRYGMYRHYFPLMALGRALRPQSHS

SEQ ID NO: 7 - protein, 625 aa, Bacillus megaterium
  source 1..625: note = BmeSHC
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 8 - protein, 642 aa, Thermosynechococcus elongatus
  source 1..642: note = TelSHC1
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSWARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQPCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTGFPCHFYLKYHYYQQHFPLTALGRYARWRNLLAT

SEQ ID NO: 9 - protein, 725 aa, Zymomonas mobilis
  source 1..725: note = ZmoSHC1
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQWARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTGFPGYGVGQTIKLDDPALSKRLLQGAELSRAFMLRYDFYRQFFPIMALSRAERLIDLNN

SEQ ID NO: 10 - protein, 658 aa, Zymomonas mobilis
  source 1..658: note = ZmoSHC2
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYWARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGINWLAQNQDEEGLWKEDYYSGGGFPRVFYLRYHGYSKYFPLWALARYRNLKKANQPIVHYGM

(19) United States
(12) Patent Application Publication
(10) Pub. No.: US 2026/0308648 A1
(43) Pub. Date: Oct. 8, 2026

(54) PROCESS FOR PRODUCING (+)-AMBERKETAL

(71) Applicant: GIVAUDAN SA, VERNIER (CH)

(72) Inventors: Thierry GRANIER, Kemptthal (CH); Eric EICHHORN, Kemptthal (CH)

(21) Appl. No.: 19/134,490

(22) PCT Filed: Dec. 13, 2023
(86) PCT No.: PCT/EP2023/085561

§ 371 Date: May 30, 2025

(30) Foreign Application Priority Data
Dec. 19, 2022 (GB) 2219135.7

(51) Int. Cl. C12P 17/06; C07C 69/145

(52) U.S. Cl. CPC C12P 17/06; C07C 69/145

(57) ABSTRACT

An enzyme-mediated method for the production of (+)-Amberketal.

### CROSS REFERENCE TO RELATED APPLICATIONS

This application is a national stage application of International Application No. PCT/EP2023/085561, filed 13 Dec. 2023, which claims priority from Great Britain Patent Application Serial No. 2219135.7, filed 19 Dec. 2022, both of which applications are incorporated herein by reference.

### REFERENCE TO ELECTRONIC SEQUENCE LISTING

The contents of the electronic sequence listing (GIVP31050A_SequenceListingXML); Size: 17,300 bytes; Date of Creation: May 7, 2026) is herein incorporated by reference in its entirety.

### TECHNICAL FIELD

The present invention generally relates to a method of making Amberketal using a squalene-hopene cyclase (SHC) enzyme. The invention further relates to compositions made by said method, the various uses of said compositions, and consumer products comprising said compositions.

### BACKGROUND

Amberketal provides a powerful and tenacious ambery and woody odour that is useful in fragrance compositions alone or in combination with other woody or ambery ingredients. Amberketal is traditionally prepared from Manool via a number of chemical transformations. However, the supply of naturally derived Manool is limited. For this reason, in recent years' new routes have been develop. It is known from WO2021/209482 that Amberketal can be biocatalytical prepared from 6-(hydroxymethyl)-10-methyl-5,9-undecadien-2-one (CAS 606493-86-1). Nevertheless, it is desirable to provide a new efficient and cost effective route to obtain Amberketal.

### SUMMARY

In accordance with a first aspect of the present invention there is provided a method of making (+)-Amberketal (a compound of formula (II)) from an acyclic acetate of formula (I) via an SHC (squalene-hopene cyclase) mediated process.

In one particular embodiment there is provided a method for making a compound of formula (II),

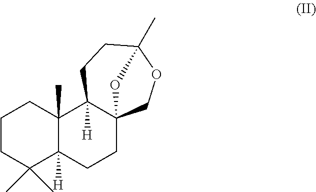

wherein the method comprises contacting a compound of formula (I)

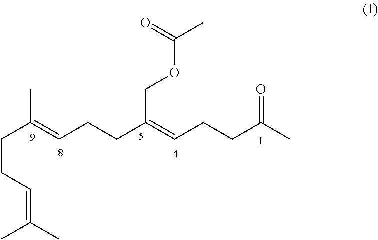 [SMILES: CC(=O)CC/C=C(/CC/C=C(\C)CCC=C(C)C)COC(C)=O]

with a squalene-hopene cyclase (SHC) enzyme, to obtain a compound of formula (III),

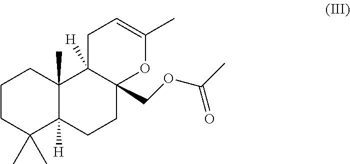 [SMILES: CC(=O)OC[C@@]12CC[C@H]3C(C)(C)CCC[C@]3(C)[C@H]1CC=C(C)O2]

followed by hydrolysis.

In a further particular embodiment there is provided a method for making a compound of formula (II),

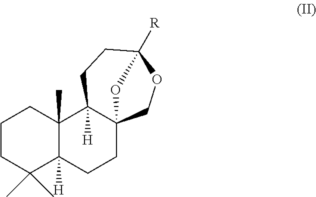

wherein the method comprises contacting a compound of formula (I)

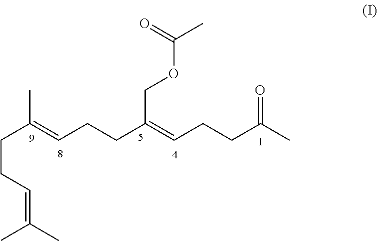 [SMILES: CC(=O)CC/C=C(/CC/C=C(\C)CCC=C(C)C)COC(C)=O]

with a squalene-hopene cyclase (SHC) enzyme and an exogenous hydrolase.

In certain embodiments of the first aspect of the present invention, the compound of formula (I) is a compound wherein the double bound between C-8 and C-9 is in E-configuration. In certain embodiments the compound of formula (I) is a compound wherein the double bond between C-8 and C-9 is in E-configuration and the double bond between C-4 and C-5 is in Z-configuration.

In accordance with a second aspect of the present invention there is provided a compound or composition obtained by or obtainable by the method of the first or second aspect of the present invention.

Certain embodiments of the present invention may provide one or more of the following advantages:
biocatalytic route to produce (+)-Amberketal;
milder reaction conditions (e.g. lower temperatures);
high selectivity;
use of alternative feed stock

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1 shows the conversion of 6,10-dimethyl-2-(4-oxopentylidene) undeca-5,9-dien-1-yl acetate (E,Z-AFA) to the compound of formula (II) ((+)-Amberketal) in the presence of an SHC and a lipase.

FIG. 2 shows the conversion of 6,10-dimethyl-2-(4-oxopentylidene) undeca-5,9-dien-1-yl acetate (E,Z-AFA) to the compound of formula (III) (AEE) in the presence of an SHC.

### SUMMARY OF THE SEQUENCES

SEQ ID NO: 1 is the amino acid sequence of wild-type Alicyclobacillus acidocaldarius SHC (AacSHC)

SEQ ID NO: 2 is the amino acid sequence of Alicyclobacillus acidocaldarius SHC enzyme variant #65 (SHC #65)

SEQ ID NO: 3 is the amino acid sequence of wild-type Gluconobacter morbifer SHC (GmoSHC)

SEQ ID NO: 4 is the amino acid sequence of wild-type Acetobacter pasteurianus SHCA (ApaSHCA)

SEQ ID NO: 5 is the amino acid sequence of wild-type Acetobacter pasteurianus SHC1 (ApaSHC1)

SEQ ID NO: 6 is the amino acid sequence of wild-type Bradyrhizobium japonicum SHC (BjaSHC)

SEQ ID NO: 7 is the amino acid sequence of wild-type Bacillus megaterium SHC (BmeSHC)

SEQ ID NO: 8 is the amino acid sequence of wild-type Thermosynechococcus elongatus SHC (TelSHC1)

SEQ ID NO: 9 is the amino acid sequence of wild-type Zymomonas mobilis SHC 1 (ZmoSHC1)

SEQ ID NO: 10 is the amino acid sequence of wild-type Zymomonas mobilis SHC2 (ZmoSHC2)

### DETAILED DESCRIPTION

The present invention is based, at least in part, on the surprising finding that (+) Amberketal can be prepared from an acyclic acetate of formula (I) via a SHC (squalene-hopene cyclase) mediated process.

Thus there is provided in a first aspect of the present invention a method of making a compound of formula (II) from a compound of formula (I) in the presence of a squalene-hopene cyclase (SHC) enzyme.

In certain embodiments there is provided a method of making a compound of formula (II),

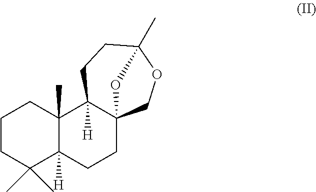

wherein the method comprises contacting a compound of formula (I)

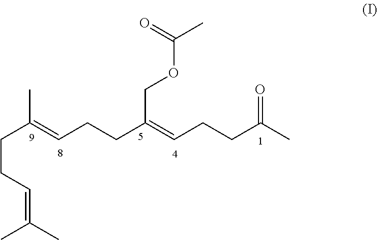 [SMILES: CC(=O)CC/C=C(/CC/C=C(\C)CCC=C(C)C)COC(C)=O]

with a squalene-hopene cyclase (SHC) enzyme, to obtain a compound of formula (III),

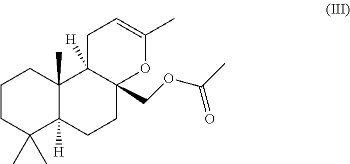 [SMILES: CC(=O)OC[C@@]12CC[C@H]3C(C)(C)CCC[C@]3(C)[C@H]1CC=C(C)O2]

followed by hydrolysis.

In certain embodiments the compound of formula (I) is a compound wherein the double bond between C-8 and C-9 is in E-configuration and the double bond between C-4 and C-5 is in Z-configuration (E,Z-compound of formula (I)).

In certain embodiments the hydrolysis takes place under acidic conditions and the acid is selected from, for example, aqueous mineral acids, including HCl, H₂SO₄, and HClO₄, aqueous sulfonic acids, including p-toluenesulfonic acid (PTSA), pyridinium p-toluenesulfonate (PPTS), camphorsulfonic acid (CSA), and methanesulfonic acid (MsOH), aqueous phosphoric and phosphonic acids, including H₃PO₄, PhPO(OH)₂, and aqueous carboxylic acids, including AcOH, HCO₂H, oxalic acid, and benzoic acid.

In certain embodiments the acid may be selected from the group consisting of acidic alumina and SiO₂.

The hydrolysis of the compound of formula (III) under acidic conditions is known in the art and described, for example, by Grant et al. (Australian Journal of Chemistry, 1994 Vol 47, 71 90).

The acid might be added to the reaction mixture after about 80 wt % or more of the compound of formula (I) was converted to the compound of formula (II). Even though the acid might be added at an earlier stage, this will result in a lower overall yield of the compound of formula (II).

In certain embodiments the acidic hydrolysis takes place in situ.

In certain embodiments the hydrolysis takes place via a transesterification in the presence of a Brønsted or Lewis acid and alcohols. The acid may be selected from Bronsted or Lewis acids ((e.g. Al(Oi-Pr)₃) and the alcohols ROH may be selected from e.g. MeOH, EtOH, etc.

Instead of acidic hydrolysis of the compound of formula (III) which is obtained when contacting the compound of formula (I) with a squalene-hopene cyclase (SHC) enzyme as described herein, the compound of formula (I) may be contacted with a mixture comprising a squalene-hopene cyclase (SHC) enzyme and an exogenous hydrolase.

Without wishing to be bound by theory, it is thought that the compound of formula (I) converts in a first step to 6-(hydroxymethyl)-10,14-dimethylpentadeca-5,9,13-trien-2-one (HFA) followed by cyclisation to the compound of formula (II).

Thus there is provided in a further embodiment a method of making a compound of formula (II),

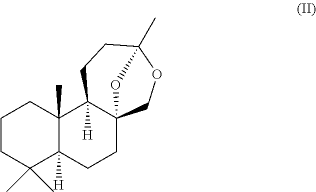

wherein the method comprises contacting a compound of formula (I)

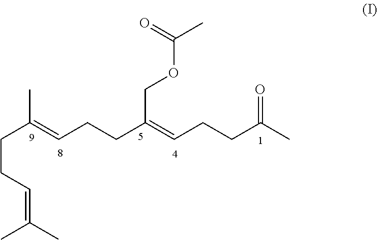 [SMILES: CC(=O)CC/C=C(/CC/C=C(\C)CCC=C(C)C)COC(C)=O]

with a squalene-hopene cyclase (SHC) enzyme and an exogenous hydrolase.

The compound of formula (I) exist in the form of four different stereoisomers, for example, as a compound of formula (I) having an E,E- or E,Z-configuration.

In certain embodiments the compound of formula (I) is a compound wherein the double bond between C-8 and C-9 is in E-configuration and the double bond between C-4 and C-5 is in Z-configuration (E,Z-compound of formula (I))

In certain embodiments, the method comprises contacting an E,Z-compound of formula (I) with a squalene-hopene cyclase (SHC) enzyme in the absence of any other stereoisomers of formula (I).

In other embodiments, the compound of formula (I) may, for example, be a mixture of stereoisomers. In certain embodiments, the mixture comprises the E,E-compound of formula (I) and one or more other stereoisomers of formula (I). In certain embodiments, the mixture comprises the E,Z-compound of formula (I) and one or more other stereoisomers of formula (I).

In certain embodiments, the method comprises contacting a mixture comprising, consisting essentially of, or consisting of E,E-compound of formula (I) and E,Z-compound of formula (I) with a SHC enzyme. In certain embodiments, the composition does not comprise any other stereoisomers of formula (I).

The weight ratio of the E,Z-compound of formula (I) to total other stereoisomers of formula (I) may, for example, be equal to or greater than about 10:90. For example, the weight ratio of the E,Z-compound of formula (I) to total other stereoisomers of formula (I) may be equal to or greater than about 20:80 or equal to or greater than about 30:70 or equal to or greater than about 40:60 or equal to or greater than about 50:50 or equal to or greater than about 60:40 or equal to or greater than about 70:30 or equal to or greater than about 80:20 or equal to or greater than about 90:10 or equal to or greater than about 95:5, or equal to or greater than about 99:1.

The weight ratio of the E,Z-compound of formula (I) to total other stereoisomers of formula (I) may, for example, be equal to or less than about 99:1. For example, the weight ratio of the E,Z-compound of formula (I) to other stereoisomers of formula (I) may be equal to or less than about 95:5 or equal to or less than about 90:10 or equal to or less than about 85:15 or equal to or less than about 80:20 or equal to or less than about 60:40.

For example, the weight ratio of the E,Z-compound of formula (I) to total other stereoisomers of formula (I) may range from about 10:90 to about 99:1 or from about 10:90 to about 90:10 or from about 20:80 to about 80:20 or from about 50:50 to about 80:20 or from about 60:40 to about 80:20.

The weight ratio of the E,Z-compound of formula (I) to the E,E-compound of formula (I) may, for example, be equal to or greater than about 10:90. For example, the weight ratio of the E,Z-compound of formula (I) to the E,E-compound of formula (II) may be equal to or greater than about 20:80 or equal to or greater than about 30:70 or equal to or greater than about 40:60 or equal to or greater than about 50:50 or equal to or greater than about 60:40 or equal to or greater than about 70:30 or equal to or greater than about 80:20 or equal to or greater than about 90:10 or equal to or greater than about 95:5, or equal to or greater than about 99:1.

The weight ratio of the E,Z-compound of formula (I) to the E,E-compound of formula (I) may, for example, be equal to or less than about 99.5:0.5. For example, the weight ratio of the E,Z-compound of formula (I) to the E,E-compound of formula (I) may be equal to or less than about 99:1 or equal to or less than about 95:5 or equal to or less than about 90:10 or equal to or less than about 85:15 or equal to or less than about 80:20 or equal to or less than about 70:30 or equal to or less than 60:40.

For example, the weight ratio of the E,Z-compound of formula (I) to the E,E-compound of formula (I) may range from about 10:90 to about 99:1 or from about 10:90 to about 90:10 or from about 20:80 to about 80:20 or from about 50:50 to about 80:20 or from about 60:40 to about 80:20.

The amount of each stereoisomer in a mixture of stereoisomers may, for example, be identified by gas chromatography or NMR spectroscopy analysis.

The number of stereoisomers of the compound of formula (I) present may influence the reaction rate. A SHC enzyme may be capable of converting an E,Z-compound of formula (I) from a complex mixture of stereoisomers of the compound of formula (I) (said mixture may include only two of the stereoisomers, for example, E,Z-compound and E,E-compound of formula (I), or it may comprise three (i.e. E,Z-, and E,E- and Z,E- or Z,Z-compound of formula (I), or even all four stereoisomers). However, a lower conversion rate may be observed, which is consistent with the view that the other stereoisomers may compete with the E,Z-compound of formula (I) for access to the SHC enzyme and thus may act as competitive inhibitors for the conversion of the E,Z-compound of formula (I) and/or also act as alternative substrates. Accordingly, the compound of formula (I) substrate may comprise an isomeric mixture of 2-4 isomers, preferably two isomers. Preferably the compound of formula (I) substrate comprises, consists essentially of or consists of an isomeric mixture of E,Z- and E,E-compound of formula (I).

To date, the compound of formula (I) has not yet been described in the literature.

Accordingly, in a second aspect of the present invention there is provided a compound of formula (I) and it use as starting material for the preparation of a compound of formula (II).

The compound of formula (I) may be synthesized following acylation conditions known to the person skilled in the art as exemplified herein below. As an acetylating agent one may use acetyl chloride (AcCl) or acetic anhydride (Ac₂O) in the presence of a base (for example, amines, such as 4-(dimethylamino)pyridine (DMAP), trialkylamine (e.g. trimethylamine) in an organic solvent, such as tetrahydrofuran (THF), 2-methyltetrahydrofuran (MeTHF), cyclohexane, PhMe, dichloromethane (DCM), and the like.

### The Compound of Formula (II)

The compound of formula (II) produced by the methods described herein may be isolated by steam extraction/distillation or organic solvent extraction using a non-water miscible solvent (to separate the reaction products and unreacted substrate from the biocatalyst which stays in the aqueous phase) followed by subsequent evaporation of the solvent to obtain a crude reaction product as determined by gas chromatography (GC) analysis. The steam extraction/distillation and organic solvent extraction methods are known to those skilled in the art.

By way of example, the resulting compound of formula (II) may be extracted from the whole reaction mixture using an organic solvent such as a non-water miscible solvent (for example toluene). Alternatively, the resulting compound of formula (II) may be extracted from the solid phase of the reaction mixture (obtained by, for example, centrifugation or filtration) using a water miscible solvent (for example ethanol) or a non-water miscible solvent (for example toluene). By way of further example, the compound of formula (II) may be present in the solid phase as crystals or in amorphous form and can be separated from the remaining solid phase (cell material or debris thereof) and the liquid phase also by means of filtration. By way of further example, at a temperature above the melting point of the compound of formula (II), the compound of formula (II) may form an oil layer on top of aqueous phase, which oil layer can be removed and collected. In order to ensure a complete recovery of compound of formula (II) after the oil layer is removed, an organic solvent may be added to the aqueous phase containing the biomass in order to extract any residual compound of formula (II) contained in, or on or about the biomass. The organic layer can be combined with the oil layer, before the whole is further processed to isolate and purify the compound of formula (II). The compound of formula (II) may be further selectively crystallised to remove by-products and any unreacted compound of formula (I) from the final product. The term “selective crystallization” refers to a process step whereby the compound of formula (II) is caused to crystallise from a solvent whilst the by-products remain dissolved in the crystallising solvent to such an extent that isolated crystalline material contains only the compound of formula (II), or if it contains any by-products, then they are present only in olfactory acceptable amounts. The selective crystallisation step may use a water miscible solvent such as ethanol or the like. The selective crystallisation of the compound of formula (II) may be influenced by the presence of unreacted compound of formula (I) and also the ratio of compound of formula (II) to the other detectable by-products. Even if only 10% conversion of the compound of formula (I) to compound of formula (II) is obtained, the selective crystallisation of the compound of formula (I) may be still possible.

Examples of suitable water miscible and non-water miscible organic solvents suitable for use in the extraction and/or selective crystallization of compound of formula (I) include but are not limited to aliphatic hydrocarbons, preferably those having 5 to 8 carbon atoms, such as pentane, cyclopentane, cyclohexane, heptane, octane or cyclooctane, aromatic hydrocarbons, such as toluene, the xylenes, chlorobenzene or, aliphatic acyclic and cyclic ethers or alcohols, preferably those having 4 to 8 carbon atoms, such as ethanol, isopropanol, diethyl ether, methyl tert.-butyl ether, ethyl tert.-butyl ether, dipropyl ether, diisopropyl ether, dibutyl ether, tetrahydrofuran, methyl-tetrahydrofuran or esters such as ethyl acetate or n-butyl acetate or ketones such as methyl isobutyl ketone or mixtures of these. The solvents which are especially preferably used are the abovementioned heptane, Methyl tert-butyl ether (also known as MTBE, tert-butyl methyl ether, tertiary butyl methyl ether, and tBME), diisopropyl ether, methyl-tetrahydrofuran, ethyl acetate and/or mixtures thereof. Preferably, a water miscible solvent such as ethanol is used for the extraction of the compound of formula (II) from the solid phase of the reaction mixture. The use of ethanol is advantageous because it is easy to handle, it is non-toxic and it is environmentally friendly.

The term “isolated” as used herein refers to a bioconversion product such as the compound of formula (II) which has been separated or purified from components which accompany it. An entity that is produced in a cellular system different from the source from which it naturally originates is “isolated”, because it will necessarily be free of components which naturally accompany it. The degree of isolation or purity can be measured by any appropriate method, e.g. gas chromatography (GC), HPLC or NMR analysis.

In some embodiments, the compound of formula (II) is isolated and purified from the obtained crude product (e.g. to a purity of at least 80%, 81%, 82%, 83%, 84%, 85%, 86%, 87%, 88%, 89%, 90%, 91%, 92%, 93%, 94%, 95%, 96%, 97%, 98%, 99%, or 100%).

Desirably, the concentration of compound of formula (II) in the reaction broth obtained by the method described herein can be from about 1 mg/l to about 20,000 mg/l (20 g/l) or higher such as from about 20 g/l to about 200 g/l or from 100-500 g/l (including 150 g/l, 250 g/l, 300 g/l, 350 g/l, 400 g/l or 450 g/l).

The compound of formula (II) as obtained by the method described herein may, for example, be in amorphous form or in crystalline form.

The compound of formula (II) contains a number of chiral carbon atoms and thus one or more stereoisomers of the compound of formula (II) may also exist, including enantiomers and diastereomers. In addition to the compound of formula (II), the products made by the methods described herein may include one or more of the stereoisomers of the compound of formula (II). The stereoisomers obtained may depend on the stereoisomers of the compound of formula (I) that are used.

For example, a compound of formula (IV) (also known as (−)-epi-8-Amberketal) may also be made in addition to the compound of formula (II),

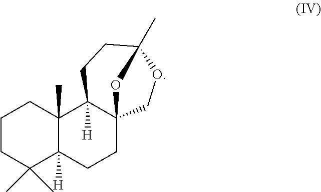

In certain embodiments, no other stereoisomers of the compound of formula (II) are made by the method or are present in the product of the method, e.g. in certain embodiments a compound of formula (IV) is not made by the method or are present in the product of the method.

The methods described herein may, for example, make a compound of formula (II) and one or more other stereoisomers of the compound of formula (II) (e.g. a compound of formula (IV)). Thus, the compositions described herein, for example the compositions obtained by or obtainable by the methods described herein may comprise a compound of formula (II) and one or more stereoisomers of the compound of formula (II) (e.g. a compound of formula (IV)).

As used herein, “making a compound of formula (II)” may be also be referred to as “producing” or “obtaining” the respective compound. It may also refer to “producing” or “obtaining” a mixture comprising, consisting essentially of, or consisting of the respective compound.

### SHC Enzyme

The method described herein use an SHC enzyme to enzymatically convert the compound of formula (I).

As used herein, the term “SHC enzyme” means both, a wild-type squalene hopene cyclase (SHC) enzyme that is naturally occurring in bacteria and variant(s) of this SHC enzyme.

The term “variant” is to be understood as a polypeptide which differs in comparison to the polypeptide from which it is derived by one or more changes in the amino acid sequence. The polypeptide from which a variant is derived is also known as the parent polypeptide. Typically, a variant is produced artificially, preferably by gene-technological means. Typically, the polypeptide from which the variant is derived is a wild-type enzyme. However, the variants usable in the present disclosure may also be derived from homologs, orthologs, or paralogs of the parent polypeptide or from artificially constructed variants. The changes in the amino acid sequence may be amino acid exchanges (substitutions), insertions, deletions, N-terminal truncations, or C-terminal truncations, or any combination of these changes, which may occur at one or several sites.

Typically, polypeptides that exhibit at least about 30% amino acid sequence identity are useful to identify conserved regions. Conserved regions of related polypeptides exhibit at least 30%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, amino acid sequence identity. In some embodiments, a conserved region exhibits at least, 70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity. The determination of sequence identity is well known to the person skilled in the art.

In one particular embodiment the wild-type squalene hopene cyclase enzyme is isolated from the thermophilic bacterium Alicyclobacillus acidocaldarius.

In a particular embodiment, the SHC enzyme (e.g. from which the SHC enzyme may be derived-wild type or variant thereof) may be the Alicyclobacillus acidocaldarius (Aac) SHC enzyme, an Acetobacter pasteurianus (Apa) SHC enzyme, a Zymomonas mobilis (Zmo) SHC enzyme, a Bradyrhizobium japonicum (Bja) SHC enzyme, a Bacillus megaterium (Bme) SHC enzyme, a Thermosynechococcus elongatus (TelSHC1) SHC enzyme, or a Gluconobacter morbifer (Gmo) SHC enzyme. In a certain embodiment, the SHC enzyme (e.g. from which the SHC enzyme may be derived-wild type or variant thereof) may be the Bacillus megaterium SHC enzyme. In a certain embodiment, the SHC enzyme (e.g. from which the SHC enzyme may be derived-wild type or variant thereof) may be the Alicyclobacillus acidocaldarius SHC enzyme. In a further embodiment, the SHC enzyme (e.g. from which the SHC enzyme may be derived-wild type or variant thereof) may be the Bradyrhizobium japonicum SHC enzyme.

For ease of reference, the designation “AacSHC” may be used to refer to the Alicyclobacillus acidocaldarius (Aac) SHC enzyme, “ApaSHC” refer to the Acetobacter pasteurianus (Apa) SHC enzyme, “ZmoSHC” may be used to refer to a Zymomonas mobilis (Zmo) SHC enzyme, “BmeSHC” may be used to refer to the Bacillus megaterium (Bme) SHC enzyme, “BjaSHC” may be used to refer to the Bradyrhizobium japonicum (Bja) SHC enzyme, “TelSHC1” refer to Thermosynechococcus elongatus (TelSHC1) SHC enzyme, and “GmoSHC” may be used to refer to the Gluconobacter morbifer (Gmo) SHC enzyme.

AacSHC, ZmoSHC and BjaSHC enzyme sequences are disclosed in BASF WO 2010/139719, US 2012/01345477A1, Seitz et al (2012) J. Molecular Catalysis B: Enzymatic 84:72-77) and Seitz (2012 PHD thesis, https://elib.uni-stuttgart.de/handle/11682/1400. Two different sequences are disclosed for ZmoSHC, referred to as ZmoSHC1 and ZmoSHC2. The GmoSHC enzyme sequence is disclosed in WO 2018/157021. Table 1 discloses sources and accession numbers of wild-type SHC enzymes.

TABLE 1
Sources and accession numbers of wild-type SHC enzymes.

| SHC Source Strain (SHC name) | Reference | Accession No. |
|---|---|---|
| Alicyclobacillus acidocaldarius | JP2009-060799 | NBRC15652 |
| (WT AacSHC) | Neumann et al Biol Chem (1986) | |
| | 367; 723-729 | |
| Zymomonas mobilis | WO2010139719 | ATCC31821 |
| (WT ZmoSHC) | (US20120135477) | PF62207_2 |
| | | Genpept |
| | | Accession No |
| | | AAV90172 |
| Zymomonas mobilis | Reipen et al (1995) Microbiology | EMBL/Genbank |
| (WT ZmoSHC) | 141: 155-161 | Accession No. |
| | | X80766 |
| Bradryhizobium japonicum | WO2010139719 | PF62207_5 |
| (WT BjaSHC) | US2012/0135477 | |
| Bacillus megaterium | WO2017/150695 (US2019078120) | WP_016763969.1 |
| (WT BmeSHC) | WO2019/045058 (US2020354754) | |
| | WO2015/033746 (US2016304911) | |
| Burkholderia ambifaria | WO2010139719 | |
| Bacillus anthracis | (US2012/0135477) | |
| Frankia alni | | |
| Rhodopseudomonas palustris | | |
| Gluconobacter morbifer | WO 2018/157021 | |
| Acetobacter pasteurianus | WO2021/110848 | ASC07046.1 |
| (WT ApaSHC1) | | |
| Acetobacter pasteurianus | WO2021/110848 | WP_003625617 |
| (WT ApaSHCA) | | |
| Thermosynechococcus | WO2021/110848 | BAC09861.1 |
| elongatus (WT TelSHC) | | |

The method for making the compound of formula (II) described herein are carried out under conditions of time, temperature, pH and solubilizing agent (if used) to provide for conversion of the compound of formula (I) to the compound of formula (II).

The method for making the compound of formula (II) may be carried out at the optimum temperature range or optimum temperature and/or the optimum pH range or optimum pH and/or solubilisation agent (if used) optimum concentration range or optimum solubilisation agent concentration for the specific enzyme used.

The pH of the reaction mixture may be in the range of 4-8, preferably, 4.5 to 6.5, more preferably 4.5-6.5 for the SHC wild-type enzyme or SHC enzyme variant considered and can be maintained by running the reaction in an appropriate buffer, or adjusting pH during the time course of the reaction. An exemplary buffer for this purpose is a citric acid buffer, or a succinic acid buffer.

The temperature may be between from about 15° C. to about 60° C., for example from about 15° C. to about 50° C. or from about 15° C. to about 45° C. or from about 30° C. to about 60° C. or from about 35° C. to about 55° C. for the SHC enzyme. The temperature can be kept constant or can be altered during the bioconversion process.

In one particular embodiment, the temperature will be increase as soon as the hydrolysis under acidic conditions is initiated. For example, the temperature may be increased from the initial ruction temperature of about 30° C.-40° C. to about 50° C. to 70° C.

It may be useful to include solubilizing agent(s) (e.g., surfactant, detergent, solubility enhancer, water miscible organic solvent and the like) in the bioconversion reaction. Examples of surfactants include but are not limited to Triton® X-100, Tween® 80, taurodeoxycholate, sodium taurodeoxycholate, sodium dodecyl sulfate (SDS), and/or sodium lauryl sulfate (SLS). In one particular embodiment SDS is used as solubilizing agent.

The method for making the compound of formula (III) disclosed herein may be carried out at the optimum temperature range or optimum temperature and/or the optimum pH range or optimum pH and/or solubilisation agent (if used) optimum concentration range or optimum solubilisation agent concentration for the specific enzyme used.

The pH of the reaction mixture may be in the range of 4-8, preferably, 4.5 to 6.5, more preferably 4.5-6.5 for the SHC wild-type enzyme or SHC enzyme variant considered and can be maintained by running the reaction in an appropriate buffer, or adjusting pH during the time course of the reaction. An exemplary buffer for this purpose is a citric acid buffer, or a succinic acid buffer.

The temperature may be between from about 15° C. to about 60° C., for example from about 15° C. to about 50° C. or from about 15° C. to about 45° C. or from about 30° C. to about 60° C. or from about 35° C. to about 55° C. for the SHC enzyme. The temperature can be kept constant or can be altered during the bioconversion process.

It may be useful to include solubilizing agent(s) (e.g., surfactant, detergent, solubility enhancer, water miscible organic solvent and the like) in the bioconversion reaction. Examples of surfactants include but are not limited to Triton® X-100, Tween® 80, taurodeoxycholate, sodium taurodeoxycholate, sodium dodecyl sulfate (SDS), and/or sodium lauryl sulfate (SLS). In one particular embodiment SDS is used as solubilizing agent.

In one particular embodiment the method described herein takes place in the presence of an SHC enzyme and an exogenous hydrolase.

It is noted that the SHC mediated process as herein described is carried out with whole cells producing the respective squalene hopene cyclase. The reaction mixtures containing whole cells contain also hydrolase enzymes of lipase and/or esterase-type, which are produced by the cells for ensuring their own metabolic purposes. These endogenous hydrolases may hydrolyse to a lesser extent the ester group of compounds serving as substrate for the SHC enzyme (e.g., compound I), or compounds resulting from the SHC cyclization reaction itself (e.g. compound III), albeit these compounds are not the substrates recognized by the endogenous hydrolases in their primary metabolic function.

An exogenous hydrolase (lipase or esterase) is therefore required to either provoke hydrolysis, or to noticeably increase the level of hydrolysis caused by the enzymes naturally occurring in the cells.

As used herein, the term “exogenous”, such as “exogenous hydrolase”, “exogenous lipase” is meant the active addition of a hydrolase, e.g., a lipase. Accordingly, the term “exogenous” explicit excludes a hydrolase which is formed in whole cells producing the squalene hopene cyclase. Alternatively, the person skilled in the art may engineer the respective SHC producing strain to produce in addition a hydrolase. Said hydrolase is as well regarded as “exogenous hydrolase” within the mean of this invention.

Wherever the method of making (+)-Amberketal (compound of formula (II) as described herein) takes place in the presence of an exogenous hydrolase, the term “hydrolase” means a family of enzymes that catalyses the hydrolysis. Said hydrolase may be any type of hydrolase from the EC 3.1 class (e.g. EC 3.1.1 class).

In one particular embodiment the exogenous hydrolase is a lipase.

A lipase is a polypeptide, having lipase activity. Their physiological function is the hydrolysis of triglycerides into di- or monoglycerides, fatty acids and glycerol. For example, a lipase may be a lipase as disclosed in Table 5. The EC number refers to Enzyme Nomenclature 1992 from NC-IUBMB, Academic Press, San Diego, Calif., including supplements 1-5 published in Eur. J. Biochem., 1994, 223:1-5; Eur. J. Biochem., 1995, 232:1-6; Eur. J. Biochem., 1996, 237:1-5; Eur. J. Biochem., 1997, 250:1-6; and Eur. J. Biochem., 1999, 264:610-650; respectively. The nomenclature is regularly supplemented and updated; see, e.g., https://iubmb.qmul.ac.uk/.

A lipase may be obtained by culturing a suitable microorganism and subsequently isolating a lipase expressed by the microorganism. Suitable microorganisms for this purpose belong to the genera Mucor, Aspergillus, Rhizopus, Rhizomucor, Pseudomonas, Candida, Humicola, Thermomyces, Burkholderia and Penicillium. Preferred species are Pseudomonas cepacia, and Pseudomonas fluorescens. A lipase that may be obtained by culturing a suitable microorganism and subsequently isolating a lipase expressed by the microorganism, may also be called a lipase from a microbial source herein.

However, lipases are also commercially available. Suppliers are, for example, Sigma Aldrich and Biocatalysts Limited (UK).

In one embodiment, the lipase belongs to the genera of Pseudomonas, for example, Pseudomonas cepacia or Pseudomonas fluorescens.

In another embodiment, the lipase may be immobilized (“immobilized lipase”). An immobilized lipase is a lipase which is adsorbed onto a carrier. A carrier is an inert, solid material. An immobilized lipase may be held in suspension in the phase wherein the reaction as described herein is performed, e.g. via agitation.

The following illustrates examples of the methods and other aspects described herein. Thus, these Examples should not be considered as limitations of the present disclosure, but are merely in place to teach how to make examples of the present disclosure.

### Examples

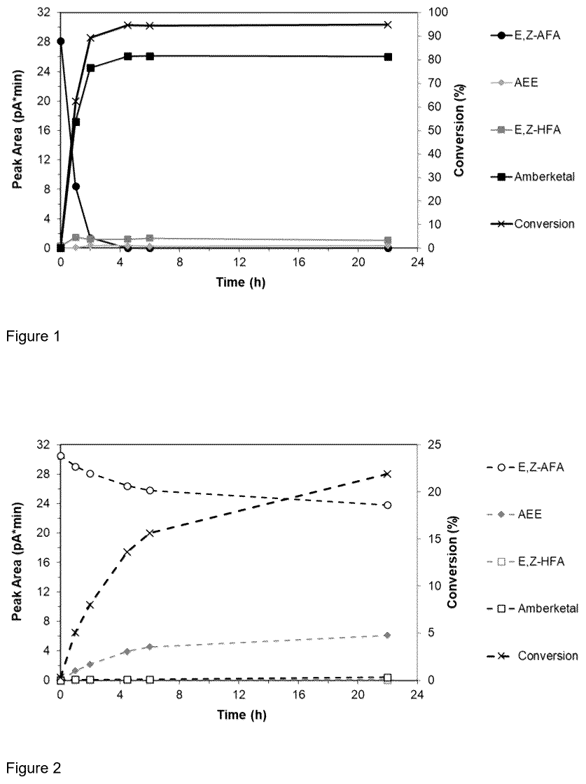

The abbreviations used in the Examples below and the FIGS. 1 and 2 have the following meaning:
AFA: 6,10-dimethyl-2-(4-oxopentylidene) undeca-5,9-dien-1-yl acetate (compound of formula (I))
(E,Z)-AFA: (2Z,5E)-6,10-dimethyl-2-(4-oxopentylidene) undeca-5,9-dien-1-yl acetate
HFA: 6-(hydroxymethyl)-10,14-dimethylpentadeca-5,9,13-trien-2-one
E,Z-HFA: (5Z,9E)-6-(hydroxymethyl)-10,14-dimethylpentadeca-5,9,13-trien-2-one
AEE: ((4aR,5aS,10aS,10bR)-(3,7,7,10a-tetramethyl-1,5,6,6a,7,8,9,10,10a, 10b-decahydro-4aH-benzo[f]chromen-4a-yl)methyl acetate (compound of formula (III))

### Example 1:(2Z,5E)-6,10-dimethyl-2-(4-oxopentylidene) undeca-5,9-dien-1-yl acetate (E,Z-acyloxyfarnesylacetone=E,Z-AFA)

A solution of (5Z,9E)-6-(hydroxymethyl)-10,14-dimethylpentadeca-5,9,13-trien-2-one (1 g, 3.3 mmol) in dichloromethane (5 ml) was treated, at room temperature, with triethylamine (0.38 g, 3.8 mmol) and acetyl chloride (0.27 ml, 3.8 mmol). The resulting mixture was stirred at room temperature during 2.5 h, treated with water (10 ml) and MTBE (10 ml) and the aq. phase extracted with MTBE (2×10 ml). The combined org. phases were washed with aq. sat. NaCl solution (10 ml), dried (MgSO₄), filtered and concentrated under reduced pressure affording 1.03 g of a crude oil that was purified by filtration over SiO₂ (elution with MTBE) leading to (2Z,5E)-6,10-dimethyl-2-(4-oxopentylidene) undeca-5,9-dien-1-yl acetate (0.94 g, 88% yield).

¹H NMR (400 MHz, CDCl₃) δ ppm 5.36 (t, J=7.3, 1H), 5.11-5.03 (m, 2H), 4.60 (s, 2H), 2.48 (t, J=7.1, 2H), 2.35 (dt, J=7.1, 7.3, 2H), 2.12 (s, 3H), 2.05 (s, 3H), 2.10-2.00 (m, 6H), 1.99-1.92 (m, 2H), 1.67 (br. s, 3H), 1.59 (br. s, 3H), 1.58 (br. s, 3H).

¹³C NMR (100 MHz, CDCl₃) δ ppm 207.87 (s, 1 C), 170.95 (s, 1 C), 135.44 (s, 1 C), 134.71 (s, 1 C), 131.25 (s, 1 C), 128.91 (d, 1 C), 124.21 (d, 1 C), 123.50 (d, 1 C), 61.74 (t, 1 C), 43.44 (t, 1 C), 39.63 (t, 1 C), 35.16 (t, 1 C), 29.86 (q, 1 C), 26.67 (t, 1 C), 26.52 (t, 1 C), 25.63 (q, 1 C), 22.01 (t, 1 C), 20.93 (q, 1 C), 17.62 (q, 1 C), 15.97 (q, 1 C).

GC-MS (EI) m/z 320 (1), 278 (1), 260 (1), 245 (1), 227 (1), 217 (3), 202 (3), 187 (2), 175 (5), 159 (6), 149 (3), 137 (4), 133 (28), 123 (11), 109 (8), 107 (10), 105 (11), 95 (10), 93 (16), 91 (10), 81 (39), 79 (11), 69 (87), 67 (13), 60 (1), 55 (11), 53 (10), 43 (100), 41 (52), 29 (3).

### Example 2: Biocatalyst Production

For squalene hopene cyclase enzyme production in Escherichia coli the gene coding for the desired squalene hopene cyclase enzyme (wt or variant) was inserted into plasmid pET-28a (+), where it is under the control of an IPTG inducible T7-promotor. The plasmid was transformed into E. coli strain BL21 (DE3) using a standard heat-shock transformation procedure.

### Cultivation Medium

The minimal medium used as default for biocatalyst production contained
10% 10× citric acid/phosphate buffer (133 g/l KH2PO4, 40 g/l (NH4)2HPO4, 17 g/l citric acid·H₂O in deionized water, with pH adjusted to 6.8 using 32% NaOH),
2.43% MgSO₄ solution (50% w/v MgSO₄·7H₂O in deionized water),
0.01% trace elements solution (50 g/l Na₂EDTA·2H₂O, 20 g/l FeSO₄·7H₂O, 3 g/l H₃BO₃, 0.9 g/l MnSO₄·2H₂O, 1.1 g/l CoCl₂, 80 g/l CuCl₂, 240 g/l NiSO₄·7H₂O, 100 g/l KI, 1.4 g/l (NH₄)6Mo₇O₂₄·4H₂O, 1 g/l ZnSO₄·7H₂O in deionized water),
0.01% Thiamin solution (2.25 g/l Thiamin·HCl in deionized water),
2% glucose solution (20% w/v glucose in deionized water).

The citric acid/phosphate buffer was first sterilized by autoclaving, the other ingredients added afterwards from sterile solutions sterilized either by autoclaving or filter-sterilization (0.2 μm).

### Fermentation

Fermentations were run in 750 ml InforsHT reactors. To the fermentation vessel was added 168 ml deionized water. The reaction vessel was equipped with all required probes (pO₂, pH, sampling, antifoam), C+N feed and sodium hydroxide bottles and autoclaved. After autoclaving is added to the reactor:

20 ml 10× phosphate/citric acid buffer
14 ml 50% glucose
0.53 ml MgSO₄ solution
2 ml (NH₄)₂SO₄ solution (50% (w/V) (NH₄)₂SO₄ in deionized water)
0.020 ml trace elements solution
0.400 ml thiamine solution
0.200 ml kanamycin solution (50 mg/ml)

The running parameters were as follows: pH=6.95, pO₂=40%, T=30° C., 300 rpm. Cascade: rpm setpoint at 300, min 300, max 1000, flow (l/min) set point 0.1, min 0, max 0.6. Antifoam control: 1:9.

A seed culture was grown in LB medium (+Kanamycin) at 37° C., 220 rpm for 8 h. The fermenter was inoculated to an OD650 nm of 0.4-0.5 from this seed culture. The fermentation was run first in batch mode for 11.5 h, where after was started the C+N feed with a feed solution (sterilized glucose solution (143 ml H₂O+35 g glucose) to which had been added after sterilization: 17.5 ml (NH₄)₂SO₄ solution, 1.8 ml MgSO₄ solution, 0.018 ml trace elements solution, 0.360 ml Thiamine solution, 0.180 ml kanamycin solution. The feed was run at a constant flow rate of approx. 4.2 ml/h. Glucose and NH₄⁺ measurements were done externally to evaluate availability of the C- and N-sources in the culture.

Cultures were grown for a total of approx. 25 hours, where they reached typically and OD_{650 nm} of 40-45. SHC production was then induced by the addition of IPTG to a concentration of 1 mM to the fermenter, and lasted for approx. 16 h at 30° C. and pO₂=20%. At the end of induction, the cells were collected by centrifugation, washed with citric acid/sodium phosphate buffer pH 5.6 and stored as pellets at 4° C. or −20° C. until further use.

### Example 3: Squalene Hopene Cyclase Screening

SHC enzymes were screened for their ability to cyclize E,Z-AFA (Example 1) in reactions containing 1 g/l substrate, cells to an OD_{650 nm} of 50 and sodium dodecylsuphate (SDS) as a surfactant. The reactions (1 ml volume) were run in citric acid/sodium phosphate buffer at pH, temperature and SDS concentration as set out in Table 2. In a negative control reaction were cells used as biocatalysts, which had not produced any SHC enzyme. The reactions were incubated in an Heidolph Synthesis 1 liquid 24 apparatus under constant agitation (800 rpm) for 22 hours, fully extracted with MTBE, and tested for their substrate and products content (GC-FID).

TABLE 2
Reaction conditions for SHC enzymes.

| | SEQ ID | Temperature | | [SDS] |
|---|---|---|---|---|
| SHC | NO | (° C.) | pH | (w/v %)¹ |
| none | | 35° C. | 5.8 | 0.050 |
| AacSHC | 1 | 50° C. | 6.0 | 0.25 |
| SHC#65 | 2 | 45° C. | 5.6 | 0.35 |
| GmoSHC | 3 | 40° C. | 5.6 | 0.038 |
| ApaSHCA | 4 | 40° C. | 6.4 | 0.050 |
| ApaSHC1 | 5 | 45° C. | 5.2 | 0.038 |
| BjaSHC | 6 | 50° C. | 5.8 | 0.025 |
| BmeSHC | 7 | 45° C. | 5.6 | 0.013 |
| TelSHC1 | 8 | 45° C. | 6.4 | 0.038 |
| ZmoSHC1 | 9 | 40° C. | 5.2 | 0.025 |
| ZmoSHC2 | 10 | 35° C. | 6.4 | 0.025 |

¹In reactions containing cells to an OD_{650 nm} of 50.

As exemplified in Example 5, (3,7,7,10a-tetramethyl-1,5,6,6a,7,8,9,10,10a, 10b-decahydro-4aH-benzo[f]chromen-4a-yl)methyl acetate (AEE) was identified as cyclization product of E,Z-AFA. E,Z-Hydroxyfarnesylacetone (E,Z-HFA) and (+)-Amberketal were also detected as reaction products whenever at minor amounts (Table 3). It was noted that in the negative control reaction with cells producing no SHC, E,Z-HFA was observed as hydrolysis product of E,Z-AFA.

TABLE 3
E,Z-AFA cyclization with SHC enzymes.

| | SEQ ID | E,Z-AFA | AEE | E,Z-HFA | (+) Amberketal |
|---|---|---|---|---|---|
| SHC | NO | conversion (%) | (% of product) | (% of product) | (% of product) |
| none | | 1.9 | 0 | 100.0 | 0 |
| AacSHC | 1 | 2.5 | 90.7 | 7.9 | 1.4 |
| SHC#65 | 2 | 16.9 | 98.2 | 0.9 | 0.9 |
| GmoSHC | 3 | 2.8 | 87.0 | 3.4 | 9.7 |
| ApaSHCA | 4 | 1.0 | 13.5 | 81.0 | 5.5 |
| ApaSHC1 | 5 | 2.6 | 86.4 | 3.7 | 9.9 |
| BjaSHC | 6 | 3.6 | 87.5 | 2.2 | 10.3 |
| BmeSHC | 7 | 40.7 | 98.6 | 0.1 | 1.3 |
| TelSHC1 | 8 | 3.2 | 80.4 | 16.5 | 3.1 |
| ZmoSHC1 | 9 | 1.7 | 80.0 | 5.1 | 15.0 |
| ZmoSHC2 | 10 | 1.5 | 20.6 | 75.4 | 4.0 |

### Example 4: Lipase Screening

Lipase enzymes were screened to identify those showing a high conversion of E,Z-AFA to E,Z-HFA. Reactions (1 ml total volume) containing 5 g/l substrate, approx. 1.25 g/l lipase were run in potassium phosphate buffer pH 7.5 at 35° C., and under constant agitation (800 rpm) in an Heidolph Synthesis 1 liquid 24 apparatus. The reactions were extracted 24 hours after start with MTBE and analyzed for their E,Z-HFA content (GC-FID). The results are shown in Table 4 below. The level of hydrolysis varies depending on the lipase used.

TABLE 4
Hydrolysis of E,Z-AFA to E,Z-HFA with lipase enzymes.

| Lipase | source | Conversion (%) |
|---|---|---|
| Candida rugosa | Sigma Aldrich 62316 | 43.5 |
| Candida antarctica CalB | Sigma Aldrich 62288 | 51.4 |
| Mucor miehei | Sigma Aldrich 62298 | 59.7 |
| Rhizopus arrhizus | Sigma Aldrich 62305 | 8.3 |
| Pseudomonas cepacia | Sigma Aldrich 62309 | 93.6 |
| Pseudomonas flurescens | Sigma Aldrich 89601 | 88.4 |
| Rhizopus niveus | Sigma Aldrich 62310 | 2.6 |
| Aspergillus sp. | Sigma Aldrich 62301 | 5.2 |
| Porcine pancreas | Sigma Aldrich L3126 | 46.3 |

### Example 5: ((4aR,5aS,10aS, 10bR)-(3,7,7,10a-tetramethyl-1,5,6,6a,7,8,9,10,10a, 10b-decahydro-4aH-benzo[f]chromen-4a-yl)methyl acetate (AEE; compound of formula (III))

40 mg of E,Z-AFA (Example 1) were cyclized in a preparative scale reaction. The reaction contained 2 g/l E,Z-AFA, 330 g/l cells that had produced wild type BmeSHC and was run at 45° C. in presence of 0.050% SDS in citric acid/Na phosphate buffer pH 5.6. Full conversion was obtained in approx. 20 hours. The reaction mixture was extracted with MTBE (3×), the MTBE fractions pooled and dried over MgSO₄, filtered and the solvent evaporated, resulting in 0.188 g crude product which was purified by microflash chromatography (MTBE/heptane) on SiO₂ yielding the title compound (6.5 mg, 16% yield).

¹H NMR (600 MHz, C₆D₆) δ ppm 4.55-4.49 (m, 2H), 4.23 (d, J=11.7, 1H), 2.44 (dt, J=3.0, 12.8, 1H), 1.79 (br. s, 3H), 1.78 (s, 3H), 1.73-0.78 (m, 12H), 0.77 (s, 3H), 0.70 (s, 3H), 0.65 (s, 3H), 0.67-0.60 (m, 1H).

¹³C NMR (150 MHz, C₆D₆) δ ppm 170.21 (s, 1 C), 148.32 (s, 1 C), 95.24 (d, 1 C), 76.70 (s, 1 C), 60.28 (t, 1 C), 56.34 (d, 1 C), 52.68 (d, 1 C), 41.94 (t, 1 C), 39.26 (t, 1 C), 36.70 (s, 1 C), 35.09 (t, 1 C), 33.39 (q, 1 C), 33.14 (s, 1 C), 21.56 (q, 1 C), 20.54 (q, 1 C), 20.30 (q, 1 C), 19.44 (t, 1 C), 18.87 (t, 1 C), 18.24 (t, 1 C), 15.60 (q, 1 C).

GC-MS (El) m/z 320 (6), 302 (1), 287 (1), 278 (1), 260 (8), 247 (1), 245 (12), 217 (17), 202 (3), 189 (9), 175 (6), 161 (5), 149 (10), 137 (21), 135 (11), 133 (11), 123 (28), 122 (30), 121 (22), 109 (20), 107 (16), 105 (14), 95 (21), 93 (22), 91 (15), 81 (24), 79 (17), 69 (22), 67 (14), 61 (1), 55 (25), 43 (100), 41 (27), 29 (5).

### Example 6: (+)-Amberketal from E,Z-AFA

Reactions were run with E,Z-AFA and E. coli cells that had produced BmeSHC with or without addition of Pseudomonas fluorescens lipase. The reactions (3 ml volume) contained 2 g/l E,Z-AFA, cells to an OD_{650 nm} of 50, and 0.125 g/l SDS. They were run in citric acid/sodium phosphate buffer pH 5.6 at 45° C. under constant agitation (350 rpm in a Radleys Carousel 12 Plus). To one reaction was added 0.5 g/l Pseudomonas fluorescens lipase.

Samples were drawn over time, extracted with MTBE and their content analyzed by GC-FID for substrate (E,Z-AFA), and products (AEE, E,Z-HFA and (+)-Amberketal).

In the absence of an exogenous lipase, E,Z-AFA was converted to AEE, only traces of E,Z-HFA and (+)-Amberketal were observed resulting from low background lipase-hydrolysis activity due to the use of whole cells. Conversion was approx. 20% in 22 hours (FIG. 2).

When lipase was added to the reaction, full conversion of E,Z-AFA was obtained in 4 to 6 hours. (+)-Amberketal appeared as the main reaction product, E,Z-HFA was observed at low levels (FIG. 1).

### Example 7: (+)-Amberketal Production from E,Z-AFA

The reaction was run as exemplified in Example 5. Full conversion of E,Z-AFA was obtained in approx. 20 hours. The reaction was then acidified to pH 2.2 by the addition of phosphoric acid. The amount of AEE decreased then over time with concomitant increase of (+)-Amberketal. Further acidification (pH 2.0) allowed for full conversion of AEE to (+)-Amberketal.

1. 6,10-dimethyl-2-(4-oxopentylidene) undeca-5,9-dien-1-yl acetate of formula (I)

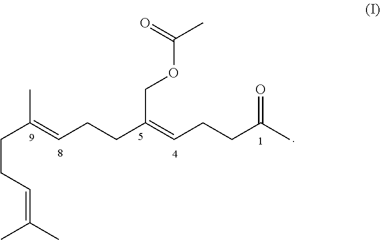 [SMILES: CC(=O)CC/C=C(/CC/C=C(\C)CCC=C(C)C)COC(C)=O]

2. The compound according to claim 1 wherein the double bound between C-8 and C-9 is in E-configuration.

3. The compound according to claim 1 wherein the double bond between C-8 and C-9 is in E-configuration and the double bond between C-4 and C-5 is in Z-configuration.

4. A method of making a compound of formula (II) from an acyclic acetate of formula (I) 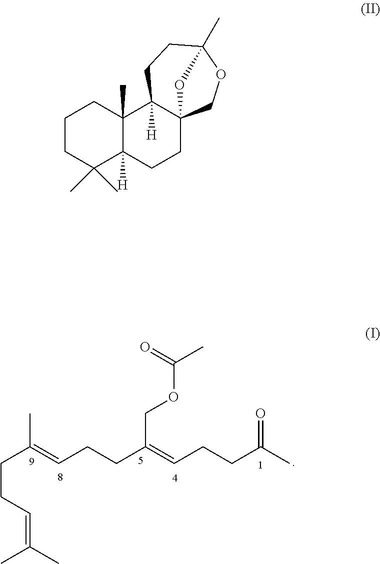
via a SHC (squalene-hopene cyclase) mediated process.

5. The A method according to claim 4 of making a compound of formula (II), 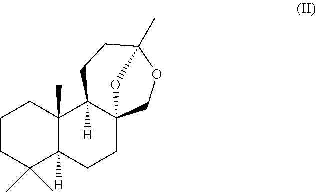
wherein the method comprises contacting a compound of formula (I) 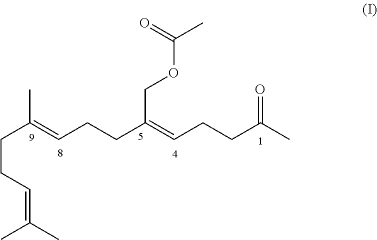 [SMILES: CC(=O)CC/C=C(/CC/C=C(\C)CCC=C(C)C)COC(C)=O]
with a squalene-hopene cyclase (SHC) enzyme, to obtain a compound of formula (III), 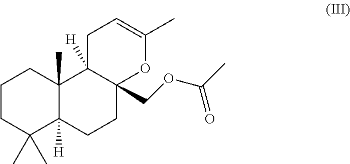 [SMILES: CC(=O)OC[C@@]12CC[C@H]3C(C)(C)CCC[C@]3(C)[C@H]1CC=C(C)O2]
followed by hydrolysis.

6. The method according to claim 5, wherein the hydrolysis occurs under acidic conditions.

7. The method according to claim 5, wherein the hydrolysis occurs under acidic conditions obtained by adding at least one acid selected from aqueous mineral acids, aqueous sulfonic acids, aqueous phosphoric and phosphonic acids, aqueous carboxylic acids, acidic alumina and SiO₂.

8. The method according to claim 4 of making a compound of formula (II), 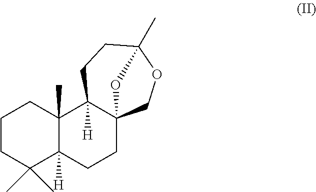
wherein the method comprises contacting a compound of formula (I) 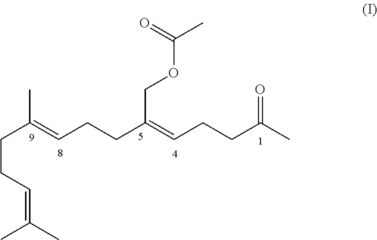 [SMILES: CC(=O)CC/C=C(/CC/C=C(\C)CCC=C(C)C)COC(C)=O]
with a squalene-hopene cyclase (SHC) enzyme and an exogenous hydrolase.

9. The method according to claim 8, wherein the exogenous hydrolase is a lipase.

10. The method of claim 4, wherein the compound of formula (I) is a compound wherein the double bond between C-8 and C-9 is in E-configuration and the double bond between C-4 and C-5 is in Z-configuration.

11. The compound according to claim 2 wherein the double bond between C-8 and C-9 is in E-configuration and the double bond between C-4 and C-5 is in Z-configuration.
